(19) United States
(12) Patent Application Publication
(10) Pub. No.: US 2013/0084254 A1
(43) Pub. Date: Apr. 4, 2013

(54) ORAL CARE COMPOSITIONS

(71) Applicants: Coralie Claudine Alonso, Bebington (GB); Alison Katharine Green, Bebington (GB); Jordan Todorov Petkov, Bebington (GB); Pierre Starck, Bebington (GB)

(72) Inventors: Coralie Claudine Alonso, Bebington (GB); Alison Katharine Green, Bebington (GB); Jordan Todorov Petkov, Bebington (GB); Pierre Starck, Bebington (GB)

(21) Appl. No.: 13/702,082

(22) PCT Filed: May 13, 2011
(86) PCT No.: PCT/EP2011/057782

§ 371 Date: Dec. 5, 2012

(30) Foreign Application Priority Data
Jun. 17, 2010 (EP) 10166300.3

(51) Int. Cl. A61K 8/64; A61K 8/19; A61K 8/25

(52) U.S. Cl. CPC A61K 8/64; A61K 8/25; A61K 8/19
USPC 424/54; 530/324

(57) ABSTRACT

The invention provides a non-aerated, foamable oral care composition comprising less than 1.5% anionic surfactant (by total weight anionic surfactant based on the total weight of the composition), abrasive cleaning agent and hydrophobin. The composition is mild to oral mucosa yet exhibits excellent foamability, texture and storage stability.

### FIELD OF THE INVENTION

The present invention relates to oral care compositions which exhibit enhanced mildness without compromising foamability, product texture or phase stability.

### BACKGROUND OF THE INVENTION

Foam is a desirable characteristic of oral care compositions such as dentifrices, since it enables the dentifrice to spread throughout the oral cavity during brushing and contact tooth surfaces thoroughly. Compositions with good foaming ability are also preferred by consumers since the foaming provides the perception that the composition is cleaning effectively.

Good foaming ability is generally achieved in oral care compositions by the use of an anionic surface active agent. Sodium lauryl sulphate (SLS) is the most commonly used anionic surfactant, and a typical dentifrice contains up to 2 or 3% of SLS (by weight based on total weight) for its foaming and surfactant action.

Anionic surface active agents such as SLS have been associated in some cases with mild adverse effects such as unpleasant flavour reactions when drinking or eating citrus shortly after tooth brushing. Accordingly, for consumers susceptible to these effects it would be desirable to reduce the content of anionic surface active agents such as SLS.

However, other surface active agents generally do not foam as well as the anionic surface active agents.

Efforts have been made in the prior art to reduce the surfactant content of a high foaming toothpaste. According to U.S. Pat. No. 4,301,141, the inclusion in a toothpaste of gelatin or a gelatinous egg white product makes it possible to significantly reduce the toothpaste surfactant content and still obtain high foaming ability.

However, attempts to reproduce toothpastes described in the specific examples of U.S. Pat. No. 4,301,141 have resulted in products which immediately phase separate on storage and which have a pronounced unpleasant “jelly-like” texture. Furthermore, the level of sodium lauryl sulphate in these products is not particularly low, ranging from 1.5 to 2% (by weight based on total weight).

It is an object of the present invention to provide oral care compositions which have a significantly reduced level of anionic surfactant compared to conventional levels, but which do not suffer from the disadvantages described above.

### SUMMARY OF THE INVENTION

The present invention provides a non-aerated, foamable oral care composition comprising less than 1.5% anionic surfactant (by total weight anionic surfactant based on the total weight of the composition), abrasive cleaning agent and hydrophobin.

The composition of the invention is mild to oral mucosa yet exhibits excellent foamability, texture and storage stability.

In another aspect the invention provides the use of hydrophobin for boosting mildness in a non-aerated, foamable oral care composition.

Hydrophobins are a group of very surface-active, fungal proteins known to self-assemble on various hydrophobic/hydrophilic interfaces. The self-assembled films coat fungal structures and mediate their attachment to surfaces. Hydrophobins have been proposed for use in cosmetics, for the purpose of surface binding. US2003/0217419 suggests that hydrophobins can be used to treat the surface of keratin materials in order to obtain a cosmetic deposit that withstands several shampoo washes. CA 2 612 458 describes a cosmetic composition containing a hydrophobin polypeptide sequence, which is alleged to bind to keratin-containing materials, mucosa or teeth.

### DETAILED DESCRIPTION OF THE INVENTION

The term “non-aerated” in the context of the present invention means a composition into which gas (i.e. air or other gas such as carbon dioxide, nitrogen, nitrous oxide, propane, butane, isobutane, dimethyl ether or mixtures thereof) has not been intentionally incorporated prior to usage by the consumer.

The term “foamable” in the context of the present invention means a composition which is capable of forming a foam in the process of usage by the consumer, such as during tooth brushing with the composition.

Anionic Surfactant

The oral care composition of the invention comprises less than 1.5% anionic surfactant (by total weight anionic surfactant based on the total weight of the composition).

Examples of anionic surfactants include the sodium, magnesium, ammonium or ethanolamine salts of C₈ to C₁₈ alkyl sulphates (for example sodium lauryl sulphate), C₈ to C₁₈ alkyl sulphosuccinates (for example dioctyl sodium sulphosuccinate), C₈ to C₁₈ alkyl sulphoacetates (such as sodium lauryl sulphoacetate), C₈ to C₁₈ alkyl sarcosinates (such as sodium lauryl sarcosinate), C₈ to C₁₈ alkyl phosphates (which can optionally comprise up to 10 ethylene oxide and/or propylene oxide units) and sulphated monoglycerides.

Mixtures of any of the above described anionic surfactants may also be used.

The total amount of anionic surfactant in compositions of the invention preferably ranges from 0 to 1.5%, more preferably from 0.25 to 1.0% by total weight anionic surfactant based on the total weight of the composition. This provides the optimum balance between mildness and foaming.

Abrasive Cleaning Agent

The oral care composition of the invention comprises abrasive cleaning agent.

Suitable abrasive cleaning agents include abrasive silicas (such as silica xerogels, hydrogels and aerogels and precipitated particulate silicas), calcium carbonates, dicalcium phosphate, tricalcium phosphate, calcined alumina, sodium and potassium metaphosphate, sodium and potassium pyrophosphates, sodium trimetaphosphate, sodium hexametaphosphate and particulate hydroxyapatite.

Calcium carbonates are a preferred class of abrasive cleaning agent in compositions of the invention. The amount of calcium carbonate in compositions of the invention generally ranges from 10% to 70%, more preferably from 20% to 50% by weight based on the total weight of the composition.

Abrasive silicas are another preferred class of abrasive cleaning agent in compositions of the invention. The amount of abrasive silica in compositions of the invention generally ranges from 2% to 20%, more preferably from 5% to 12% by weight based on the total weight of the composition.

Mixtures of any of the above described abrasive cleaning agents may also be used.

The total amount of abrasive cleaning agent in compositions of the invention will depend on the particular agent (or agents) used, but suitably ranges from 3 to 75% by total weight abrasive cleaning agent based on the total weight of the composition.

Hydrophobin

The oral care composition of the invention comprises at least one hydrophobin.

Hydrophobins are a well-defined class of proteins (Wessels, 1997, Adv. Microb. Physio. 38: 1-45; Wosten, 2001, Annu Rev. Microbiol. 55: 625-646) capable of self-assembly at a hydrophobic/hydrophilic interface, and having a conserved sequence:

(SEQ ID No. 1)
Xₙ-C-X₅₋₉-C-C-X₁₁₋₃₉-C-X₈₋₂₃-C-X₅₋₉-C-C-X₆₋₁₈-
C-Xₘ

where X represents any amino acid, and n and m independently represent an integer. Typically, a hydrophobin has a length of up to 125 amino acids. The cysteine residues (C) in the conserved sequence are part of disulphide bridges. In the context of this invention, the term hydrophobin has a wider meaning to include functionally equivalent proteins still displaying the characteristic of self-assembly at a hydrophobic-hydrophilic interface resulting in a protein film, such as proteins comprising the sequence:

(SEQ ID No. 2)
Xₙ-C-X₁₋₅₀-C-X₀₋₅-C-X₁₋₁₀₀-C-X₁₋₁₀₀-C-X₁₋₅₀-C-
X₀₋₅-C-X₁₋₅₀-C-Xₘ

or parts thereof still displaying the characteristic of self-assembly at a hydrophobic-hydrophilic interface resulting in a protein film. In accordance with the definition of this invention, self-assembly can be detected by adsorbing the protein to Teflon and using Circular Dichroism to establish the presence of a secondary structure (in general, α-helix) (De Vocht et al., 1998, Biophys. J. 74: 2059-68).

The formation of a film can be established by incubating a Teflon sheet in the protein solution followed by at least three washes with water or buffer (Wosten et al., 1994, Embo. J. 13: 5848-54). The protein film can be visualised by any suitable method, such as labelling with a fluorescent marker or by the use of fluorescent antibodies, as is well established in the art. m and n typically have values ranging from 0 to 2000, but more usually m and n in total are less than 100 or 200. The definition of hydrophobin in the context of this invention includes fusion proteins of a hydrophobin and another polypeptide as well as conjugates of hydrophobin and other molecules such as polysaccharides.

Hydrophobins identified to date are generally classed as either class I or class II. Both types have been identified in fungi as secreted proteins that self-assemble at hydrophobic-hydrophilic interfaces into amphipathic films.

Hydrophobin-like proteins have also been identified in filamentous bacteria, such as Actinomycete and Streptomyces sp. (WO01/74864; Talbot, 2003, Curr. Biol, 13: R696-R698). These bacterial proteins by contrast to fungal hydrophobins, may form only up to one disulphide bridge since they may have only two cysteine residues. Such proteins are an example of functional equivalents to hydrophobins having the consensus sequences shown in SEQ ID Nos. 1 and 2, and are within the scope of this invention.

The hydrophobins can be obtained by extraction from native sources, such as filamentous fungi, by any suitable process. For example, hydrophobins can be obtained by culturing filamentous fungi that secrete the hydrophobin into the growth medium or by extraction from fungal mycelia with 60% ethanol. It is particularly preferred to isolate hydrophobins from host organisms that naturally secrete hydrophobins. Preferred hosts are hyphomycetes (e.g. Trichoderma), basidiomycetes and ascomycetes. Particularly preferred hosts are food grade organisms, such as Cryphonectria parasitica which secretes a hydrophobin termed cryparin (MacCabe and Van Alfen, 1999, App. Environ. Microbiol 65: 5431-5435).

Alternatively, hydrophobins can be obtained by the use of recombinant technology. For example host cells, typically micro-organisms, may be modified to express hydrophobins and the hydrophobins can then be isolated and used in accordance with the present invention. Techniques for introducing nucleic acid constructs encoding hydrophobins into host cells are well known in the art. More than 34 genes coding for hydrophobins have been cloned, from over 16 fungal species (see for example WO96/41882 which gives the sequence of hydrophobins identified in Agaricus bisporus; and Wosten, 2001, Annu. Rev. Microbiol. 55: 625-646). Recombinant technology can also be used to modify hydrophobin sequences or synthesise novel hydrophobins having desired/improved properties.

Typically, an appropriate host cell or organism is transformed by a nucleic acid construct that encodes the desired hydrophobin. The nucleotide sequence coding for the polypeptide can be inserted into a suitable expression vector encoding the necessary elements for transcription and translation and in such a manner that they will be expressed under appropriate conditions (e.g. in proper orientation and correct reading frame and with appropriate targeting and expression sequences). The methods required to construct these expression vectors are well known to those skilled in the art.

A number of expression systems may be used to express the polypeptide coding sequence. These include, but are not limited to, bacteria, fungi (including yeast), insect cell systems, plant cell culture systems and plants all transformed with the appropriate expression vectors. Preferred hosts are those that are considered food grade—‘generally regarded as safe’ (GRAS).

Suitable fungal species, include yeasts such as (but not limited to) those of the genera Saccharomyces, Kluyveromyces, Pichia, Hansenula, Candida, Schizo saccharomyces and the like, and filamentous species such as (but not limited to) those of the genera Aspergillus, Trichoderma, Mucor, Neurospora, Fusarium and the like.

The sequences encoding the hydrophobins are preferably at least 80% identical at the amino acid level to a hydrophobin identified in nature, more preferably at least 95% or 100% identical. However, persons skilled in the art may make conservative substitutions or other amino acid changes that do not reduce the biological activity of the hydrophobin. For the purpose of the invention these hydrophobins possessing this high level of identity to a hydrophobin that naturally occurs are also embraced within the term “hydrophobins”.

Hydrophobins can be purified from culture media or cellular extracts by, for example, the procedure described in WO01/57076 which involves adsorbing the hydrophobin present in a hydrophobin-containing solution to surface and then contacting the surface with a surfactant, such as Tween 20, to elute the hydrophobin from the surface. See also Collen et al., 2002, Biochim Biophys Acta. 1569: 139-50; Calonje et al., 2002, Can. J. Microbiol. 48: 1030-4; Askolin et al., 2001, Appl Microbiol Biotechnol. 57: 124-30; and De Vries et al., 1999, Eur J Biochem. 262: 377-85.

Typically, the hydrophobin is in an isolated form, typically at least partially purified, such as at least 10% pure, based on weight of solids. By “isolated form”, we mean that the hydrophobin is not added as part of a naturally-occurring organism, such as a mushroom, which naturally expresses hydrophobins. Instead, the hydrophobin will typically either have been extracted from a naturally-occurring source or obtained by recombinant expression in a host organism.

Hydrophobin proteins can be divided into two classes: Class I, which are largely insoluble in water, and Class II, which are readily soluble in water.

Preferably, the hydrophobins chosen are Class II hydrophobins. More preferably the hydrophobins used are Class II hydrophobins such as HFBI, HFBII, HFBIII, or Cerato ulmin.

The hydrophobin can be from a single source or a plurality of sources e.g. a mixture of two or more different hydrophobins.

The total amount of hydrophobin in compositions of the invention will generally be at least 0.001%, more preferably at least 0.005 or 0.01%, and generally no greater than 2% by total weight hydrophobin based on the total weight of the composition.

Product Form

A preferred type of product form in the context of the present invention is a dentifrice. The term “dentifrice” denotes formulations which are used to clean the surfaces of the oral cavity. The dentifrice is an oral composition that is not intentionally swallowed for purposes of systemic administration of therapeutic agents, but is retained in the oral cavity for a sufficient time to contact substantially all of the dental surfaces and/or mucosal tissues for purposes of oral activity. Preferably the dentifrice is suitable for application with a toothbrush and is rinsed off after use. Preferably the dentifrice is in the form of a paste or a gel (or a combination thereof).

A dentifrice composition according to the invention will generally contain further ingredients to enhance performance and/or consumer acceptability such as water, humectant, and binder or thickening agent.

For example, the dentifrice will usually contain a liquid phase in an amount of from 40 to 99% by weight based on the total weight of the dentifrice. Such a liquid phase typically comprises water and a humectant in various relative amounts, with the amount of water generally ranging from 10 to 45% by weight (based on the total weight of the dentifrice) and the amount of humectant generally ranging from 30 to 70% by weight (based on the total weight of the dentifrice). Typical humectants include glycerol, sorbitol, polyethylene glycol, polypropylene glycol, propylene glycol, xylitol (and other edible polyhydric alcohols), hydrogenated partially hydrolyzed polysaccharides and mixtures thereof.

Furthermore, the dentifrice will usually contain a binder or thickening agent in an amount of from 0.5 to 10% by weight based on the total weight of the dentifrice. Suitable binders or thickening agents include carboxyvinyl polymers (such as polyacrylic acids cross-linked with polyallyl sucrose or polyallyl pentaerythritol), hydroxyethyl cellulose, hydroxypropyl cellulose, water soluble salts of cellulose ethers (such as sodium carboxymethyl cellulose and sodium carboxymethyl hydroxyethyl cellulose), natural gums (such as carrageenan, gum karaya, guar gum, xanthan gum, gum arabic, and gum tragacanth), finely divided silicas, hectorites, colloidal magnesium aluminum silicates and mixtures thereof.

Optional Ingredients

Flavouring agents are generally used in oral care compositions (such as dentifrices) at levels up to about 5% by weight based on the total weight of the composition. Commonly used flavouring agents are peppermint oil, spearmint oil, oil of wintergreen and mixtures thereof. A number of other flavouring agents have been suggested for use in oral products including sassafras, clove, sage, eucalyptus, marjoram, cinnamon, lemon and orange.

Mixtures of any of the above described flavouring agents may also be used.

Advantageously, we have found that in compositions of the invention, the level of flavouring agent may be reduced without significant loss of flavour impact.

Accordingly, the total amount of flavouring agent in compositions of the invention preferably ranges from 0 to 1.5% by total weight flavouring agent based on the total weight of the composition. More preferably the total amount of flavouring agent ranges from 0.1 to 1.0% by total weight flavouring agent based on the total weight of the composition. This provides the optimum balance between formulation cost and flavour impact.

Compositions of the present invention may also contain further optional ingredients customary in the art, such as fluoride ion sources, anticalculus agents, buffers, sweetening agents, colouring agents, opacifying agents, preservatives, antisensitivity agents and antimicrobial agents.

The invention is further illustrated with reference to the following, non-limiting Examples.

### EXAMPLES

Objective

A study was carried out to compare formulations according to the present invention with formulations according to U.S. Pat. No. 4,301,141, which describes the use of gelatin or gelatin hydrolysate to produce mild foaming toothpastes.

Formulations

Toothpastes were prepared having ingredients as follows:

### Examples 1 and 2

### According to the Invention

| | Example 1 | Example 2 |
|---|---|---|
| Ingredient | (% w/w) | (% w/w) |
| Sorbitol | 65.4 | 45.0 |
| Sodium saccharin | 0.3 | 0.2 |
| Polyethylene glycol 1500 | 2.0 | 2.0 |
| Sodium fluoride | 0.2 | 0.3 |
| Abrasive silica | 8.5 | 8.0 |
| Thickening silica | 9.0 | 10.0 |
| Sodium carboxymethyl cellulose | 0.6 | 0.7 |
| Titanium dioxide | — | 1.0 |
| Zinc citrate | — | 2.0 |
| Hydrophobin* | 0.1 | 0.1 |
| Sodium lauryl sulphate | 0.75 | 0.75 |
| Flavour | 0.6 | 0.6 |
| Water | to 100 | to 100 |

### Comparative Examples A and B

### According to Examples 1 and 2 Respectively of U.S. Pat. No. 4,301,141

| | Example A | Example B |
|---|---|---|
| Ingredient | (% w/w) | (% w/w) |
| Sorbitol | 12.0 | 12.0 |
| Calcium carbonate | 25.0 | 25.0 |
| Thickening silica | 2.0 | 2.0 |
| Sodium carboxymethyl cellulose | 0.8 | 1.0 |
| Sodium salt of p-hydroxybenzoic | 0.2 | 0.2 |
| acid methyl ester | | |
| Sodium lauryl sulphate | 2.0 | 1.5 |
| Sodium myristoyl taurate | 0.5 | 0.5 |
| Gelatin | 3.0 | — |
| Gelatin hydrolysate | — | 3.5 |
| Flavour | 2.0 | 2.0 |
| Water | to 100 | to 100 |

### Example 3

### According to the Invention

| | Example 3 |
|---|---|
| Ingredient | (% w/w) |
| Sorbitol | 12.0 |
| Calcium carbonate | 25.0 |
| Thickening silica | 2.0 |
| Sodium carboxymethyl cellulose | 0.8 |
| Sodium salt of p-hydroxybenzoic | 0.2 |
| acid methyl ester | |
| Sodium lauryl sulphate | 0.75 |
| Flavour | 0.6 |
| Hydrophobin* | 0.1 |
| Water | to 100 |

[*The specific hydrophobin used was Class II Hydrophobin HFBII, obtained from VTT Biotechnology, Finland. It had been purified from Trichoderma reesei essentially as described in WO00/58342 and Linder et al., 2001, Biomacromolecules 2: 511-517.]

Foaming Evaluation

Samples of each toothpaste were foamed by taking 2 g of the paste in 30 ml sterilin, diluting it with 4 mL of de-ionised water and vigorously shaking by hand for 90 seconds.

Results and Conclusions

It was noted that both Comparative Example A and Comparative Example B immediately phase separated on storage. It was also observed that the texture of these formulations was slimy and “jelly-like”.

By contrast, no stability or textural negatives were observed for any of Examples 1 to 3 according to the invention.

The foaming results are shown below in Table 1.

TABLE 1

| | Foam height as measured from |
|---|---|
| Formulation | the bottom of the vial (cm) |
| Example 1 | 67 |
| Example 2 | 39 |
| Example 3 | 73 |
| Comparative Example A | 25 |
| Comparative Example B | 25 |

It can be seen that all of Examples 1 to 3 produced significantly more foam than either Comparative Example A or Comparative Example B.

1. A non-aerated, foamable oral care composition comprising less than 1.5% anionic surfactant (by total weight anionic surfactant based on the total weight of the composition), abrasive cleaning agent and hydrophobin.

2. A non-aerated, foamable oral care composition according to claim 1, where the hydrophobin is a Class II hydrophobin.

3. A non-aerated, foamable oral care composition according to claim 2, where the Class II hydrophobin is HFBI, HFBII, or a mixture thereof.

4. An oral care composition according to claim 1, in which the amount of anionic surfactant ranges from 0.25 to 1.0% by total weight anionic surfactant based on the total weight of the composition.

5. An oral care composition according to claim 1, in which the abrasive cleaning agent is selected from abrasive silicas, calcium carbonates and mixtures thereof.

6. An oral care composition according to claim 1, in which the amount of abrasive cleaning agent ranges from 3 to 75% by total weight abrasive cleaning agent based on the total weight of the composition.

7. An oral care composition according to claim 1, in which the amount of hydrophobin ranges from 0.01% to 2% by total weight hydrophobin based on the total weight of the composition.

8. An oral care composition according to claim 1, which is in the form of a dentifrice and which comprises water, humectant, and binder or thickening agent.

9. An oral care composition according to claim 1, which further comprises a flavouring agent in an amount ranging from 0.1 to 1.0% by total weight flavouring agent based on the total weight of the composition.

10. The use of hydrophobin for boosting mildness in a non-aerated, foamable oral care composition.
